(19) United States
(12) Patent Application Publication
(10) Pub. No.: US 2025/0214975 A1
(43) Pub. Date: Jul. 3, 2025

(54) PEPTIDYL FLAVOR MODIFIERS

(71) Applicant: Ohio State Innovation Foundation, Columbus, OH (US)

(72) Inventors: Devin PETERSON, Columbus, OH (US); Christopher SIMONS, Dublin, OH (US); Brianne LINNE, Philadelphia, PA (US); Edisson TELLO CAMACHO, Columbus, OH (US)

(21) Appl. No.: 18/867,144

(22) PCT Filed: May 19, 2023
(86) PCT No.: PCT/US2023/022867

§ 371 Date: Nov. 19, 2024

Related U.S. Application Data
(60) Provisional application No. 63/343,745, filed on May 19, 2022.

(51) Int. Cl. C07D 403/14; A23C 9/156; A23F 5/46; A23L 27/00

(52) U.S. Cl. CPC C07D 403/14; A23C 9/156; A23F 5/465; A23L 27/88

(57) ABSTRACT

Disclosed herein are flavor modifying peptidyl compounds. The compounds may be used to improve the flavor, including mouthfeel, of a variety of consumable products, including coffee beverages and other coffee products.

### CROSS-REFERENCE TO RELATED APPLICATION

This application claims the benefit of U.S. Provisional Application 63/343,745, filed May 19, 2022, the contents of which are hereby incorporated in their entirety.

### FIELD OF THE INVENTION

The invention is directed to compounds and compositions useful to modulate the flavors, for instance in consumables such as foods and beverages. In an embodiment, the compounds and compositions are useful to modify and improve body, mouthfeel, chalkiness, and the like in coffee products.

### BACKGROUND

The production, trade, and consumption of coffee has changed in this current “third wave of coffee” era that has moved from a pure commodity to a specialty product. A leading driver of this trend is the demand for higher flavor quality coffee. A common practice in the coffee industry to assess the flavor quality of coffee beans (brew) is using the ‘cup score’ method of the Specialty Coffee Association (SCA). Evaluated by certified Q-graders, the SCA quality score is composed of ten sensory metrics including fragrance/aroma, acidity, body, flavor, sweetness, clean cup, balance, aftertaste, uniformity, and overall impression. This method was developed to provide consistency of the quality rating and supports criteria for establishing retail price. However, a lack of knowledge on the chemical drivers of coffee quality remains as only a few studies have related cup score to flavor chemistry.

The chemical composition of coffee products is known to consist of a complex mixture of thousands of compounds endogenous to the green coffee beans or generated during post-harvest fermentation or roasting steps. Thermal generation of flavor during roasting is known to be highly dependent on the green bean composition and the degree of roasting which impact the flavor of the final brew. Several factors from farm to cup are known to influence coffee flavor quality such as species/cultivars, geographical origin, green bean processing method, roasting and storage.

Chemical predictors of coffee quality, in both green coffee beans and brew, have been investigated. Some work has been done to establish correlations between non-volatile compounds such as sugars, amino acids, phenolic compounds and fatty acids on the quality of green and roasted coffee beans. Similarly, in coffee brew, the volatile aroma composition has been primarily studied and drivers of quality have been explored. For example, high amounts of unsaturated aldehydes such as (E,E)-2,4-nonadienal and (E,Z)-2,4-heptadienal have been associated with high quality coffee brews. Conversely, high levels of 2-phenylacetaldehyde, 2-methyl-5-propylpyrazine were associated with lower quality coffee.

However more understanding of compounds that impact coffee quality and specifically the SCA cup score is needed. There remains a need for compositions and methods for improving coffee quality, especially for converting lower quality coffee into higher quality coffee. There remains a need for compositions and methods for improving the body, mouthfeel, chalkiness, and similar aspects in coffee.

In accordance with the purposes of the disclosed materials and methods, as embodied and broadly described herein, the disclosed subject matter, in one aspect, relates to compounds, compositions and methods of making and using compounds and compositions.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

The details of one or more embodiments are set forth in the descriptions below. Other features, objects, and advantages will be apparent from the description and from the claims.

### BRIEF DESCRIPTION OF THE FIGURES

FIG. 1 depicts R-index values calculated during sensory evaluation in coffee (N=6, 18 evaluations; left panel) and water (N=6, 17 evaluations; right panel). The dashed line indicates critical value which must be exceeded for sample to be considered distinct from control. Statistical significance determined using published R-index critical values for given number of evaluations (*p≤0.05).

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions, Wiley Interscience, New York, 1981; Wilen et al., Tetrahedron 33:2725 (1977); Eliel, E. L. Stereochemistry of Carbon Compounds, McGraw-Hill, NY, 1962; and Wilen, S. H., Tables of Resolving Agents and Optical Resolutions p. 268, E. L. Eliel, Ed., Univ. of Notre Dame Press, Notre Dame, IN 1972. The invention additionally encompasses compounds as individual isomers substantially free of other isomers, and alternatively, as mixtures of various isomers.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched hydrocarbon group having a specified range of carbon atoms (e.g., a “C₁₋₁₆ alkyl” can have from 1 to 16 carbon atoms). In some embodiments, an alkyl group has 1 to 9 carbon atoms (“C₁₋₉ alkyl”). An alkyl group can be saturated or unsaturated, i.e., an alkenyl or alkynyl group as defined herein. Unless specified to the contrary, an “alkyl” group includes both saturated alkyl groups and unsaturated alkyl groups.

In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “alkylenyl” refers to a divalent radical of a straight-chain, cyclic, or branched saturated hydrocarbon group having a specified range of carbon atoms (e.g., a “C₁₋₁₆ alkyl” can have from 1 to 16 carbon atoms). An example of alkylenyl is a methylene (—CH₂—). An alkylenyl can be substituted as described above for an alkyl.

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “hydroxyalkyl” is a substituted alkyl group, wherein one or more of the hydrogen atoms are independently replaced by a hydroxyl. In some embodiments, the hydroxyalkyl moiety has 1 to 8 carbon atoms (“C₁₋₈ hydroxyalkyl”). In some embodiments, the hydroxyalkyl moiety has 1 to 6 carbon atoms (“C₁₋₆ hydroxyalkyl”). In some embodiments, the hydroxyalkyl moiety has 1 to 4 carbon atoms (“C₁₋₄ hydroxyalkyl”). In some embodiments, the hydroxyalkyl moiety has 1 to 3 carbon atoms (“C₁₋₃ hydroxyalkyl”). In some embodiments, the hydroxyalkyl moiety has 1 to 2 carbon atoms (“C₁₋₂ hydroxyalkyl”).

The term “alkoxy” refers to an alkyl group, as defined herein, appended to the parent molecular moiety through an oxygen atom. In some embodiments, the alkoxy moiety has 1 to 8 carbon atoms (“C₁₋₈ alkoxy”). In some embodiments, the alkoxy moiety has 1 to 6 carbon atoms (“C₁₋₆ alkoxy”). In some embodiments, the alkoxy moiety has 1 to 4 carbon atoms (“C₁₋₄ alkoxy”). In some embodiments, the alkoxy moiety has 1 to 3 carbon atoms (“C₁₋₃ alkoxy”). In some embodiments, the alkoxy moiety has 1 to 2 carbon atoms (“C₁₋₂ alkoxy”). Representative examples of alkoxy include, but are not limited to, methoxy, ethoxy, propoxy, 2-propoxy, butoxy and tert-butoxy.

The term “haloalkoxy” refers to a haloalkyl group, as defined herein, appended to the parent molecular moiety through an oxygen atom. In some embodiments, the alkoxy moiety has 1 to 8 carbon atoms (“C₁₋₈ haloalkoxy”). In some embodiments, the alkoxy moiety has 1 to 6 carbon atoms (“C₁₋₆ haloalkoxy”). In some embodiments, the alkoxy moiety has 1 to 4 carbon atoms (“C₁₋₄ haloalkoxy”). In some embodiments, the alkoxy moiety has 1 to 3 carbon atoms (“C₁₋₃ haloalkoxy”). In some embodiments, the alkoxy moiety has 1 to 2 carbon atoms (“C₁₋₂ haloalkoxy”). Representative examples of haloalkoxy include, but are not limited to, difluoromethoxy, trifluoromethoxy, and 2,2,2-trifluoroethoxy.

The term “alkoxyalkyl” is a substituted alkyl group, wherein one or more of the hydrogen atoms are independently replaced by an alkoxy group, as defined herein. In some embodiments, the alkoxyalkyl moiety has 1 to 8 carbon atoms (“C₁₋₈ alkoxyalkyl”). In some embodiments, the alkoxyalkyl moiety has 1 to 6 carbon atoms (“C₁₋₆ alkoxyalkyl”). In some embodiments, the alkoxyalkyl moiety has 1 to 4 carbon atoms (“C₁₋₄ alkoxyalkyl”). In some embodiments, the alkoxyalkyl moiety has 1 to 3 carbon atoms (“C₁₋₃ alkoxyalkyl”). In some embodiments, the alkoxyalkyl moiety has 1 to 2 carbon atoms (“C₁₋₂ alkoxyalkyl”).

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and 1 or more heteroatoms within the parent chain (“heteroC₁₋₁₈ alkyl”). In some embodiments, a heteroalkyl group is a saturated group having 1 to 16 carbon atoms and 1 or more heteroatoms within the parent chain (“heteroC₁₋₁₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

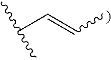

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl,” “cycloalkyl,” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₄ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like.

Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃.14 carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₆). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

As used herein, the term “heterocyclyl” refers to an aromatic (also referred to as a heteroaryl), unsaturated, or saturated cyclic hydrocarbon that includes at least one heteroatom in the cycle. For example, the term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, aziridinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofurany 1, tetrahydrothiopheny 1, dihydrothiopheny 1, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR—, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(Rh^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}(═O)(R^{aa})₂, —NR^{bb}P(═O)(OR^{cc})₂—NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₂, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X—is a counterion; or two geminal hydrogens on a carbon atom are replaced with the group ═O, ═S, ═NN(R^{bb})₂, —NNR^{bb}C(═O)R^{aa}, —NNR^{bb}C(═O)OR^{aa}, —NNR^{bb}S(═O)₂R^{aa}, ═NR or ═NOR^{cc}; each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{aa}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{dd} is, independently, selected from halogen, —CN, —NO₂, —N₃, —SO₂H, —SO₃H, —OH, —OR^{ee}, —ON(R^{f})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR)N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₄₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion; each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; each instance of R^{f} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{f} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃, —C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb})₂)₂, wherein X⁻, R^{aa}, R^{bb} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, NHC(═O)N(R^{bb})₂, —NHC(═NR^{bb})N(R^{bb})₂, —NHSO₂R^{aa}, —NHP(═O)(OR)₂, and —NHP(═O)(N(R^{bb})₂)₂, wherein R^{aa}, R^{bb}, and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})², —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₂ and —N(R^{bb})₃⁺X⁻, wherein R^{bb} and X—are as defined herein. The term “sulfonyl” refers to a group selected from —SO₂N(R^{bb})₂, —SO₂R^{aa}, and SO₂OR^{aa}, wherein R^{aa} and R^{bb} are as defined herein.

The term “sulfinyl” refers to the group —S(═O)R^{aa}, wherein R^{aa} is as defined herein. The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or dialkylamino, mono- or di-heteroalkylamino, mono- or di-arylamino, or mono- or diheteroarylamino; or two R^{X1} groups taken together form a 5- to 6-membered heterocyclic ring.

Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (e.g., —C(═O)R^{a}), carboxylic acids (e.g., —CO₂H), aldehydes (CHO), esters (e.g., —CO₂R^{aa}, —C(═O)SR^{aa}, —C(═S)SR^{aa}), amides (e.g., —C(═O)N(R^{bb})₂, C(═O)NR^{bb}SO₂R^{aa}, —C(═S)N(R^{bb})₂, and imines (e.g., —C(═NR^{bb})R^{aa}, —C(═NR^{bb})OR^{aa}), C(═NR^{bb})N(R^{bb})₂, wherein R^{aa} and R^{bb} are as defined herein.

The term “oxo” refers to the group ═O, and the term “thiooxo” refers to the group ═S.

The term “cyano” refers to the group —CN.

The term “azide” refers to the group —N₃.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{ee})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{aa}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or a 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc}, and R^{dd} are as defined herein.

As used herein, an “amino acid residue” refers to an amino acid incorporated into a peptide chain. By way of example, the following tripeptide:

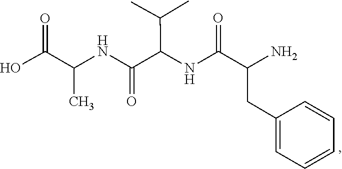 [SMILES: CC(NC(=O)C(NC(=O)C(N)Cc1ccccc1)C(C)C)C(=O)O]

is composed (from left to right) of an alanine residue, a valine residue, and a phenylalanine residue.

The terms “peptide”, “polypeptide”, and “peptidyl” are used interchangeably herein, and refer to a polymeric form of amino acids of any length, which can include coded and non-coded amino acids, chemically or biochemically modified or derivatized amino acids, and polypeptides having modified peptide backbones.

As used herein, the term “chirality” refers to the “D” and “L” isomers of amino acids or amino acid residues. In generic form, an α-L-amino acid has the following configuration:

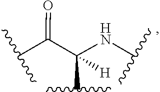 [SMILES: *N[C@@H](*)C(*)=O]

while an α-D-amino acid will have the opposite configuration. The “u” designation indicates that there is a single carbon atom between the carboxylate and amine functional groups In a “β” amino acid, there are two carbon atoms between the carboxylate and amine functional groups, in a “T” amino acid, there are three carbon atoms, etc. Reference to an amino acid, a D-amino acid, an L-amino acid, and the like should be understood to refer to α-amino acids unless specified to the contrary.

As used herein, “amino acid side chain” refers to the substituent(s) bonded to the carbon chain linking the carboxylate and amine functional group. Exemplary side chains are depicted below

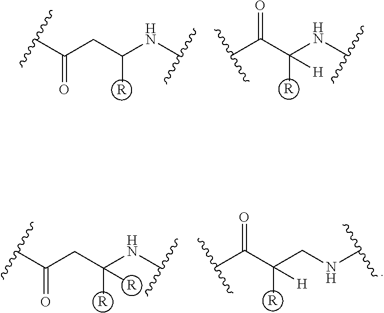

A “natural” amino acid refers to an amino acid that are incorporated into proteins during translation. There are 20 canonical amino acids in the genetic code, along with selenocysteine and pyrrolysine. As used herein, a natural amino acid can be in either the L- or D-configuration, and reference to an amino acid without specifying the configuration should be understood to be inclusive of both. A natural amino acid is an α-amino acid, and has a side chain selected from H, CH₃, CH(CH₃)₂, CH₂CH(CH₃)₂, CH(CH₃)CH₂CH₃, CH₂OH, CH(OH)CH₃, CH₂C(O)NH₂, CH₂CH₂C(O)NH₂, CH₂SH, CH₂CH₂SCH₃, CH₂COOH, CH₂CH₂COOH, CH₂CH₂CH₂CH₂NH₂, CH₂CH₂CH₂NHC(NH)NH₂, CH₂SeH, or a group having the formula:

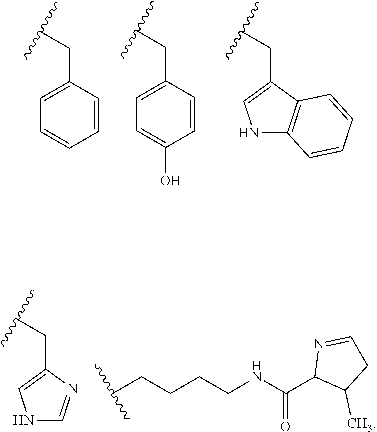

Proline, which is a naturally occurring amino acid, has a side chain that forms a five atom ring with the amine functional group.

The term “non-natural amino acid” (or “unnatural amino acid”) refers to an organic compound that is a congener of a natural amino acid in that it has an amine (—NH₂) group on one end and a carboxylic acid (—COOH) group on the other end but the side chain or backbone is modified. A non-natural amino acid has a side chain different than that found in a natural amino acid.

Amino acids can be designated by a three-letter or one-letter code, for example as shown in the table below:

| Amino Acid | Abbreviations* |
|---|---|
| alanine | Ala (A) |
| allosoleucine | AIle |
| arginine | Arg (R) |
| asparagine | Asn (N) |
| aspartic acid | Asp (D) |
| cysteine | Cys (C) |
| glutamic acid | Glu (E) |
| glutamine | Gln (Q) |
| glycine | Gly (G) |
| histidine | His (H) |
| isoleucine | Ile (I) |
| leucine | Leu (L) |
| lysine | Lys (K) |
| Methionine | Met (M) |
| napthylalanine | Nal(Φ) |
| phenylalanine | Phe (F) |
| proline | Pro (P) |
| selenocysteine | Sec (U) |
| serine | Ser (S) |
| threonine | Thr (T) |
| tyrosine | Tyr (Y) |
| tryptophan | Trp (W) |
| valine | Val (V) |
| Phenylglycine | Phg |
| Propargylglycine | Pra |

As used herein, an amino acid code with a capital letter refers to the L-amino acid. A D-amino acid is described when the code is present as a lower case letter, or as a three letter code composed entirely of lower case letters.

As used herein, a chemical bond depicted: [symbol] represents either a single, double, or triple bond, valency permitting. By way of example,

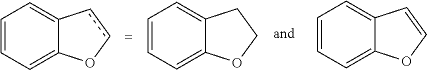

An electron-withdrawing group is a functional group or atom that pulls electron density towards itself, away from other portions of the molecule, e.g., through resonance and/or inductive effects. Exemplary electron-withdrawing groups include F, Cl, Br, I, NO₂, CN, SO₂R, SO₃R, SO₂NR₂, C(O)R^{1a}; C(O)OR, and C(O)NR₂ (wherein R is H or an alkyl, aryl, heteroaryl, cycloalkyl, heterocyclyl group) as well as alkyl group substituted with one or more of those group

An electron-donating group is a functional group or atom that pushes electron density away from itself, towards other portions of the molecule, e.g., through resonance and/or inductive effects. Exemplary electron-donating groups include unsubstituted alkyl or aryl groups, OR and N(R)₂ and alkyl groups substituted with one or more OR and N(R)₂ groups.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

Some compounds disclosed herein may exist as one or more tautomers. Tautomers are interconvertible structural isomers that differ in the position of one or more protons or other labile atom. By way of example:

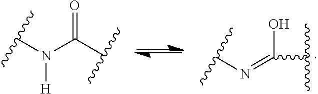

The prevalence of one tautomeric form over another will depend both on the specific chemical compound as well as its local chemical environment. Unless specified to the contrary, the depiction of one tautomeric form is inclusive of all possible tautomeric forms.

Unless stated to the contrary, a substituent drawn without explicitly specifying the point of attachment indicates that the substituent may be attached at any possible atom. For example, in a benzofuran depicted as:

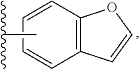

the substituent may be present at any one of the six possible carbon atoms.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

Compounds disclosed herein may be provided in the form of physiologically acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. A physiologically acceptable salt refers to counterions which are not toxic at relevant concentrations.

Disclosed herein are flavor modifying compounds of Formula (1):

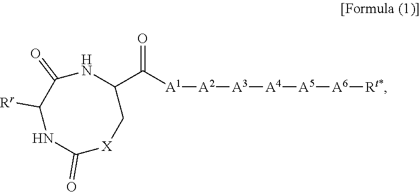

and physiologically acceptable salts thereof, wherein:
R^{t*} is OH or NH₂;
X is null, CH₂, O, S, or Se;
R has the formula:

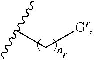

wherein nᵣ is 0, 1, 2, 3, 4, 5, or 6, and G^{r} is H, C₁₋₆alkyl, C₃₋₈cycloalkyl, C₆₋₁₀aryl, C₁₋₁₀heteroaryl, OH, SH, SCH₃, COOH, CONH₂, NH₂, NHC(═NH)NH₂, COOH, CONH₂, or CH(OH)CH₃;
A¹ is an amino acid;
A² is an amino acid;
A³ is null or an amino acid;
A⁴ is null or an amino acid;
A⁵ is null or an amino acid; and
A⁶ is null or an amino acid.

In certain embodiments, A¹ can be an L-amino acid, while in other embodiments, A¹ is a D-amino acid. In certain preferred embodiments, A¹ can be a naturally occurring L-amino acid. In some embodiments, A¹ can be an alanine residue, alloisoleucine residue, arginine residue, asparagine residue, aspartic acid residue, cysteine residue, glutamic acid residue, glutamine residue, glycine residue, histidine residue, isoleucine residue, leucine residue, lysine residue, methionine residue, pyrrololysine residue, naphthylalanine residue, phenylalanine residue, proline residue, selenocysteine residue, serine residue, threonine residue, tyrosine residue, tryptophan residue, valine residue, phenylglycine residue, or a propargylglycine residue.

In certain embodiments, A² can be an L-amino acid, while in other embodiments, A² is a D-amino acid. In certain preferred embodiments, A² can be a naturally occurring L-amino acid. In some embodiments, A² can be an alanine residue, alloisoleucine residue, arginine residue, asparagine residue, aspartic acid residue, cysteine residue, glutamic acid residue, glutamine residue, glycine residue, histidine residue, isoleucine residue, leucine residue, lysine residue, methionine residue, pyrrololysine residue, naphthylalanine residue, phenylalanine residue, proline residue, selenocysteine residue, serine residue, threonine residue, tyrosine residue, tryptophan residue, valine residue, phenylglycine residue, or a propargylglycine residue.

In certain embodiments, A³ can be an L-amino acid, while in other embodiments, A³ is a D-amino acid. In certain preferred embodiments, A³ can be a naturally occurring L-amino acid. In some embodiments, A³ can be an alanine residue, alloisoleucine residue, arginine residue, asparagine residue, aspartic acid residue, cysteine residue, glutamic acid residue, glutamine residue, glycine residue, histidine residue, isoleucine residue, leucine residue, lysine residue, methionine residue, pyrrololysine residue, naphthylalanine residue, phenylalanine residue, proline residue, selenocysteine residue, serine residue, threonine residue, tyrosine residue, tryptophan residue, valine residue, phenylglycine residue, or a propargylglycine residue. In other embodiments however, A³ is null.

In certain embodiments, A⁴ can be an L-amino acid, while in other embodiments, A⁴ is a D-amino acid. In certain preferred embodiments, A⁴ can be a naturally occurring L-amino acid. In some embodiments, A⁴ can be an alanine residue, alloisoleucine residue, arginine residue, asparagine residue, aspartic acid residue, cysteine residue, glutamic acid residue, glutamine residue, glycine residue, histidine residue, isoleucine residue, leucine residue, lysine residue, methionine residue, pyrrololysine residue, naphthylalanine residue, phenylalanine residue, proline residue, selenocysteine residue, serine residue, threonine residue, tyrosine residue, tryptophan residue, valine residue, phenylglycine residue, or a propargylglycine residue. In other embodiments however, A⁴ is null.

In certain embodiments, A⁵ can be an L-amino acid, while in other embodiments, A⁵ is a D-amino acid. In certain preferred embodiments, A⁵ can be a naturally occurring L-amino acid. In some embodiments, A⁵ can be an alanine residue, alloisoleucine residue, arginine residue, asparagine residue, aspartic acid residue, cysteine residue, glutamic acid residue, glutamine residue, glycine residue, histidine residue, isoleucine residue, leucine residue, lysine residue, methionine residue, pyrrololysine residue, naphthylalanine residue, phenylalanine residue, proline residue, selenocysteine residue, serine residue, threonine residue, tyrosine residue, tryptophan residue, valine residue, phenylglycine residue, or a propargylglycine residue. In other embodiments however, A⁵ is null.

In certain embodiments, A⁶ can be an L-amino acid, while in other embodiments, A⁶ is a D-amino acid. In certain preferred embodiments, A⁶ can be a naturally occurring L-amino acid. In some embodiments, A⁵ can be an alanine residue, alloisoleucine residue, arginine residue, asparagine residue, aspartic acid residue, cysteine residue, glutamic acid residue, glutamine residue, glycine residue, histidine residue, isoleucine residue, leucine residue, lysine residue, methionine residue, pyrrololysine residue, naphthylalanine residue, phenylalanine residue, proline residue, selenocysteine residue, serine residue, threonine residue, tyrosine residue, tryptophan residue, valine residue, phenylglycine residue, or a propargylglycine residue. In other embodiments however, A⁶ is null.

In certain embodiments, A⁵ and A⁶ are both null. In some preferred embodiments, A⁴, A⁵ and A⁶ are each null. In other preferred embodiments, A³, A⁴, A⁵ and A⁶ are each null.

In some embodiments, A¹ is a hydrophobic amino acid residue. Exemplary hydrophobic amino acid residues include tryptophan, alanine, valine, isoleucine, leucine, methionine, naphthylalanine, phenylalanine, tyrosine, and glycine. In certain embodiments, the hydrophobic residue is substituted one or more times by a fluorine atom. In some preferred embodiments, A¹ is a tryptophan, naphthylalanine, phenylalanine, or tyrosine residue, especially a tryptophan residue.

In some embodiments, A² is an ionizable amino acid residue. As used here, an ionizable amino acid residue is one having a side chain with one or more ionizable functional groups, e.g., a basic amine such as found in lysine and histidine, or a carboxylic acid such as found in glutamic acid and aspartic acid. In some embodiments, A² is a histidine, arginine, lysine, aspartic acid, or glutamine acid residue, especially a lysine, arginine, or histidine residue, and even more especially a histidine residue.

In certain embodiments nᵣ is 0 or 1, and G is H, C₁₋₆alkyl, C₃₋₈cycloalkyl, C₆₋₁₀aryl, or C₁₋₁₀heteroaryl.

G^{r} can be C₆₋₁₀aryl. Exemplary C₆₋₁₀aryl groups include, but are not limited to, unsubstituted phenyl, substituted phenyl like hydroxyphenyl (e.g., 4-hydroxyphenyl, 3-hydroxyphenyl, 2-hydroxyphenyl), fluorophenyl, carboxyphenyl, aminophenyl, unsubstituted naphthyl such as naphth-1-yl or naphth-2-yl, naphthyls substituted one or more times with fluorine, hydroxy, alkoxy, or amino, and the like.

G^{r} can be C₁₋₁₀heteroaryl. Exemplary C₁₋₁₀heteroaryl groups include, but are not limited to, pyridinyl, e.g., pyridin-2-yl, pyridine-3-yl, pyridin-4-yl, or pyridinyl, indolinyl, e.g., indole-1-yl, indole-2-yl, indole-3-yl, indole-4-yl, indole-5-yl, indole-6-yl, indole-7-yl, and imidazole, e.g., imidazol-1-yl, imidazol-2-yl, imidazol-4-yl, and imidazol-5-yl. Such C₁₋₁₀heteroaryl groups may be unsubstituted or substituted one or more times by groups such as fluorine, hydroxy, alkoxy, or amino, and the like.

G^{r} can be C₁₋₆alkyl. Exemplary C₁₋₆alkyl groups include, but are not limited to, methyl, ethyl, n-propyl, isopropyl, n-butyl, isobutyl, sec-butyl, tert-butyl, n-pentyl, isopentyl, and the like. Such C₁₋₆alkyl groups may be unsubstituted or substituted one or more times by groups such as fluorine, hydroxy, alkoxy, or amino, and the like.

In some embodiments, the flavor modifying compound can be a compound of Formula (2):

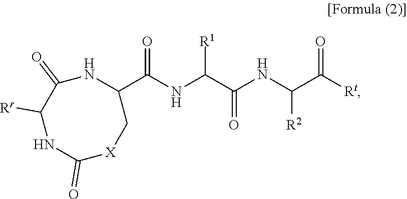

or a physiologically acceptable salt thereof, wherein:
R^{t} is —R^{t*}, -A³-R^{t*}, -A³-A⁴-R^{t*}, -A³-A⁴-R^{t*}, -A³-A⁴-A⁵-R^{t*}, and -A³-A⁴-A⁵-A⁶-R^{t*};
X is null, CH₂, O, S, or Se;
R^{r} has the formula:

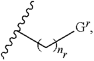

wherein nᵣ is 0, 1, 2, 3, 4, 5, or 6, and G^{r} is H, C₁₋₆alkyl, C₃₋₈cycloalkyl, C₆₋₁₀aryl, C₁₋₁₀heteroaryl, OH, SH, SCH₃, COOH, CONH₂, NH₂, NHC(═NH)NH₂, COOH, CONH₂, or CH(OH)CH₃;
R¹ has the formula:

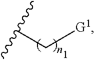

wherein n₁ is 0, 1, 2, 3, 4, 5, or 6, and G¹ is H, C₁₋₆alkyl, C₃₋₈cycloalkyl, C₆₋₁₀aryl, C₁₋₁₀heteroaryl, OH, SH, SCH₃, COOH, CONH₂, NH₂, NHC(═NH)NH₂, COOH, CONH₂, or CH(OH)CH₃; and
R² has the formula:

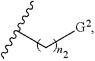

wherein n2 is 0, 1, 2, 3, 4, 5, or 6, and G² is H, C₁₋₆alkyl, C₃₋₈cycloalkyl, C₆₋₁₀aryl, C₁₋₁₀heteroaryl, OH, SH, SCH₃, COOH, CONH₂, NH₂, NHC(═NH)NH₂, COOH, CONH₂, or CH(OH)CH₃.

The disclosed compounds can exist in a variety of stereochemical configurations, for example:

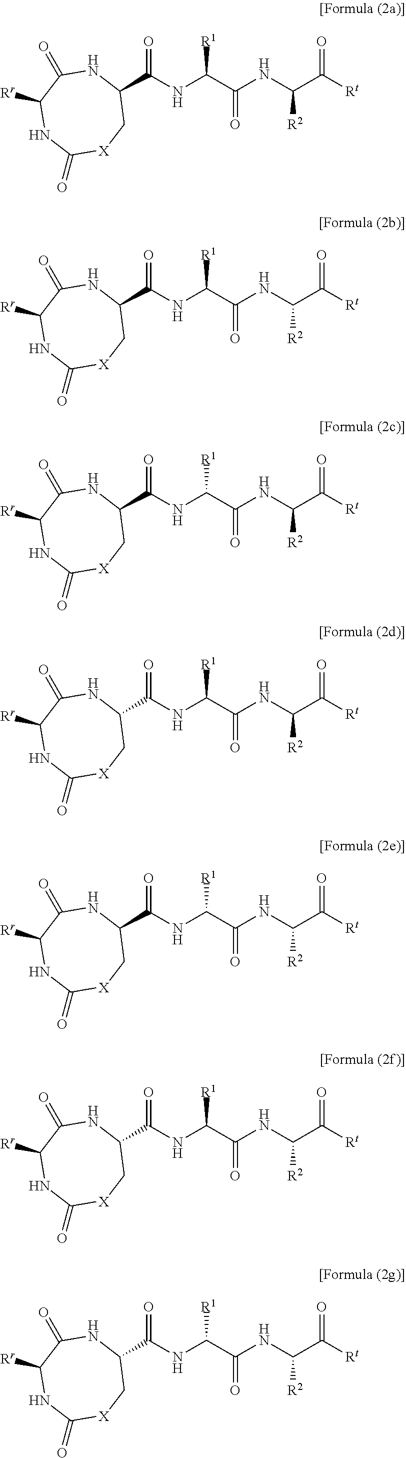

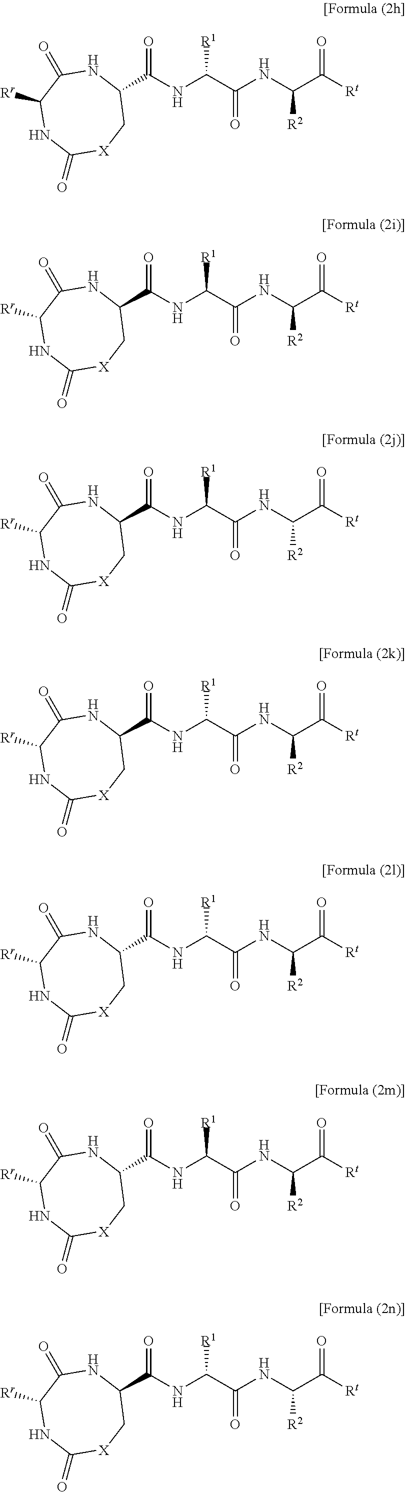

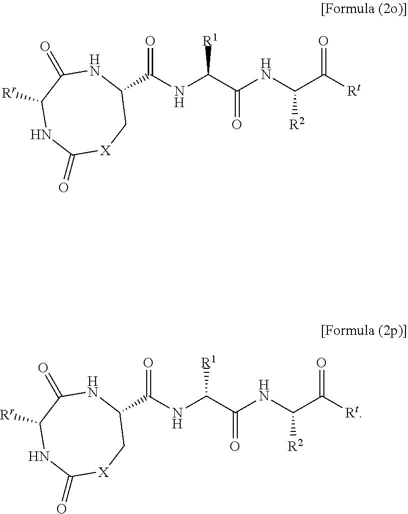

In certain embodiments, the flavor modifying compound can be predominantly a single stereoisomer of Formula 2.

In some embodiments, the flavor modifying compound is a compound of Formula (2a) in an amount of at least 75 mol %, at least 85 mol %, at least 90 mol %, at least 95 mol %, or at least 99 mol %, relative to any other compounds of Formula (2).

In some embodiments, the flavor modifying compound is a compound of Formula (2b) in an amount of at least 75 mol %, at least 85 mol %, at least 90 mol %, at least 95 mol %, or at least 99 mol %, relative to any other compounds of Formula (2).

In some embodiments, the flavor modifying compound is a compound of Formula (2c) in an amount of at least 75 mol %, at least 85 mol %, at least 90 mol %, at least 95 mol %, or at least 99 mol %, relative to any other compounds of Formula (2).

In some embodiments, the flavor modifying compound is a compound of Formula (2d) in an amount of at least 75 mol %, at least 85 mol %, at least 90 mol %, at least 95 mol %, or at least 99 mol %, relative to any other compounds of Formula (2).

In some embodiments, the flavor modifying compound is a compound of Formula (2e) in an amount of at least 75 mol %, at least 85 mol %, at least 90 mol %, at least 95 mol %, or at least 99 mol %, relative to any other compounds of Formula (2).

In some embodiments, the flavor modifying compound is a compound of Formula (2f) in an amount of at least 75 mol %, at least 85 mol %, at least 90 mol %, at least 95 mol %, or at least 99 mol %, relative to any other compounds of Formula (2).

In some embodiments, the flavor modifying compound is a compound of Formula (2g) in an amount of at least 75 mol %, at least 85 mol %, at least 90 mol %, at least 95 mol %, or at least 99 mol %, relative to any other compounds of Formula (2).

In some embodiments, the flavor modifying compound is a compound of Formula (2h) in an amount of at least 75 mol %, at least 85 mol %, at least 90 mol %, at least 95 mol %, or at least 99 mol %, relative to any other compounds of Formula (2).

In some embodiments, the flavor modifying compound is a compound of Formula (2i) in an amount of at least 75 mol %, at least 85 mol %, at least 90 mol %, at least 95 mol %, or at least 99 mol %, relative to any other compounds of Formula (2).

In some embodiments, the flavor modifying compound is a compound of Formula (2j) in an amount of at least 75 mol %, at least 85 mol %, at least 90 mol %, at least 95 mol %, or at least 99 mol %, relative to any other compounds of Formula (2).

In some embodiments, the flavor modifying compound is a compound of Formula (2k) in an amount of at least 75 mol %, at least 85 mol %, at least 90 mol %, at least 95 mol %, or at least 99 mol %, relative to any other compounds of Formula (2).

In some embodiments, the flavor modifying compound is a compound of Formula (2l) in an amount of at least 75 mol %, at least 85 mol %, at least 90 mol %, at least 95 mol %, or at least 99 mol %, relative to any other compounds of Formula (2).

In some embodiments, the flavor modifying compound is a compound of Formula (2m) in an amount of at least 75 mol %, at least 85 mol %, at least 90 mol %, at least 95 mol %, or at least 99 mol %, relative to any other compounds of Formula (2).

In some embodiments, the flavor modifying compound is a compound of Formula (2n) in an amount of at least 75 mol %, at least 85 mol %, at least 90 mol %, at least 95 mol %, or at least 99 mol %, relative to any other compounds of Formula (2).

In some embodiments, the flavor modifying compound is a compound of Formula (2o) in an amount of at least 75 mol %, at least 85 mol %, at least 90 mol %, at least 95 mol %, or at least 99 mol %, relative to any other compounds of Formula (2).

In some embodiments, the flavor modifying compound is a compound of Formula (2p) in an amount of at least 75 mol %, at least 85 mol %, at least 90 mol %, at least 95 mol %, or at least 99 mol %, relative to any other compounds of Formula (2).

In some embodiments, n₁ can be 1; in some embodiments, n₂ is 1. In some preferred embodiments, n₁ and n2 are both 1.

In some preferred embodiments, G¹ can be H, C₁₋₆alkyl, C₃₋₈cycloalkyl, C₆₋₁₀aryl, or C₁₋₁₀heteroaryl. Exemplary groups for G¹ include:

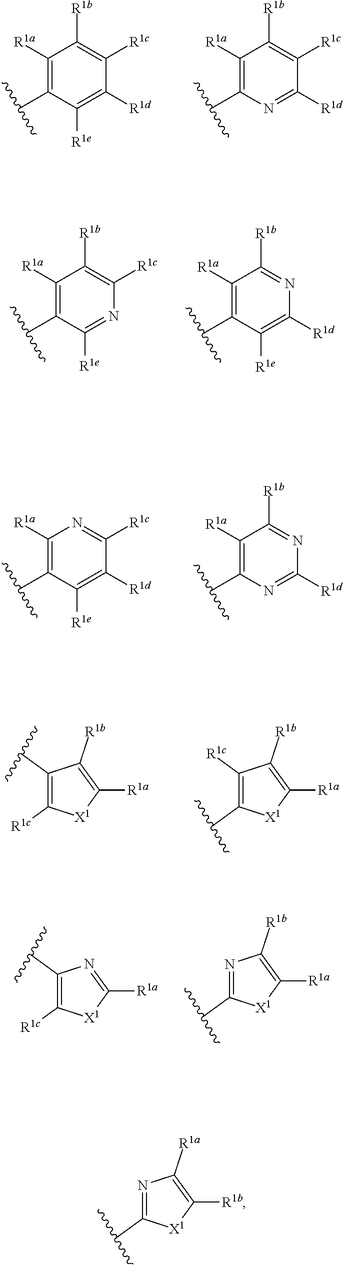

wherein
X¹ is selected from O, S, and NH;
R^{1a} is selected from H, F, Cl, Br, I, OH, COOH, C₁₋₆alkyl, OC₁₋₆alkyl;
R^{1b} is selected from H, F, Cl, Br, I, OH, COOH, C₁₋₆alkyl, OC₁₋₆alkyl;
R^{1c} is selected from H, F, Cl, Br, I, OH, COOH, C₁₋₆alkyl, OC₁₋₆alkyl;
R^{1d} is selected from H, F, Cl, Br, I, OH, COOH, C₁₋₆alkyl, OC₁₋₆alkyl;
R^{1e} is selected from H, F, Cl, Br, I, OH, COOH, C₁₋₆alkyl, OC₁₋₆alkyl;
wherein any two or more of R^{1a}, R^{1b}, R^{1c}, R^{1d}, and R^{1e} may together form a ring.

In some preferred embodiments, G² can be H, C₁₋₆alkyl, C₃₋₈cycloalkyl, C₆₋₁₀aryl, or C₁₋₁₀heteroaryl. Exemplary groups for G² include:

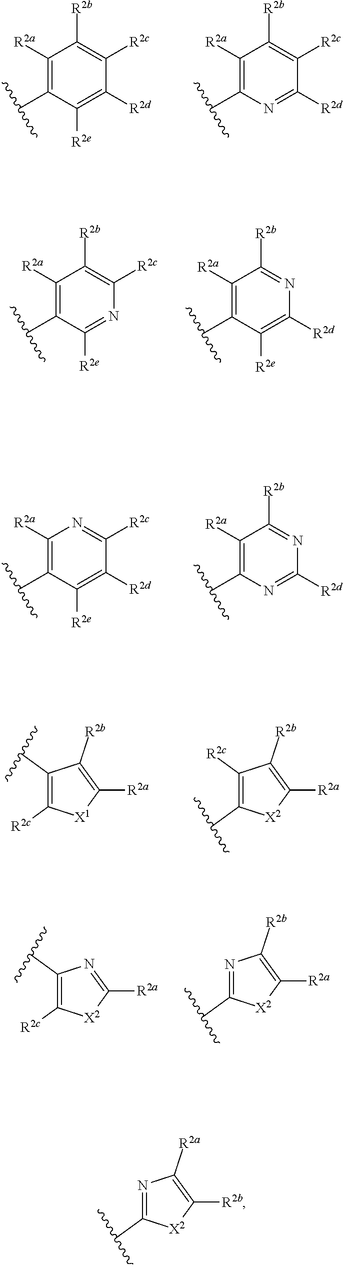

wherein
X² is selected from O, S, and NH;
R^{2a} is selected from H, F, Cl, Br, I, OH, COOH, C₁₋₆alkyl, OC₁₋₆alkyl;
R^{2b} is selected from H, F, Cl, Br, I, OH, COOH, C₁₋₆alkyl, OC₁₋₆alkyl;
R^{2c} is selected from H, F, Cl, Br, I, OH, COOH, C₁₋₆alkyl, OC₁₋₆alkyl;
R^{2d} is selected from H, F, Cl, Br, I, OH, COOH, C₁₋₆alkyl, OC₁₋₆alkyl;
R^{2e} is selected from H, F, Cl, Br, I, OH, COOH, C₁₋₆alkyl, OC₁₋₆alkyl;
wherein any two or more of R^{2a}, R^{2b}, R^{2c}, R^{2d}, and R^{2e} may together form a ring.

In some preferred embodiments, G¹ can be a group having formula:

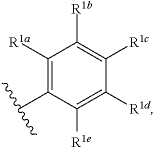

where R^{1a} and R^{1b} together form a ring, for example:

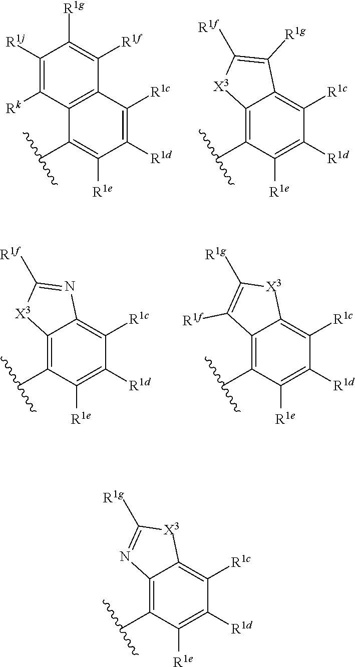

X³ is selected from O, S, and NH;
R^{1f} is selected from H, F, Cl, Br, I, OH, COOH, C₁₋₆alkyl, OC₁₋₆alkyl;
R^{1g} is selected from H, F, Cl, Br, I, OH, COOH, C₁₋₆alkyl, OC₁₋₆alkyl;
R^{1h} is selected from H, F, Cl, Br, I, OH, COOH, C₁₋₆alkyl, OC₁₋₆alkyl; and
R¹ⁱ is selected from H, F, Cl, Br, I, OH, COOH, C₁₋₆alkyl, OC₁₋₆alkyl.

In other embodiments, G¹ can be a group having the formula:

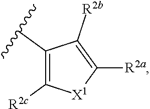

where R^{1a} and R^{1b} together form a ring, for example:

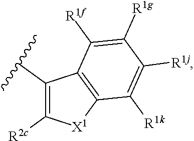

R^{1f} is selected from H, F, Cl, Br, I, OH, COOH, C₁₋₆alkyl, OC₁₋₆alkyl;
R^{1g} is selected from H, F, Cl, Br, I, OH, COOH, C₁₋₆alkyl, OC₁₋₆alkyl;
R^{1h} is selected from H, F, Cl, Br, I, OH, COOH, C₁₋₆alkyl, OC₁₋₆alkyl; and
R¹ⁱ is selected from H, F, Cl, Br, I, OH, COOH, C₁₋₆alkyl, OC₁₋₆alkyl.

In some preferred embodiments, n₁ is 1, and G¹ is an indole ring, for example an indole-3-yl group. This combination results in an R¹ group having the formula:

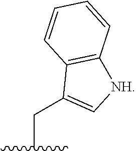 [SMILES: *Cc1c[nH]c2ccccc12]

In some preferred embodiments, n₂ is 1, and G² is an imidazole ring, for example a imidazole-4-yl group. This combination results in an R² group having the formula:

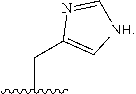 [SMILES: *Cc1c[nH]cn1]

In some embodiments, the flavor modifying compound can be a compound of Formula (3):

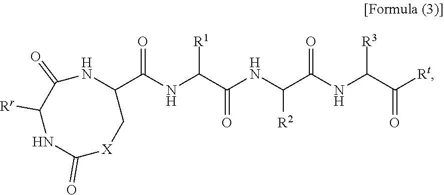

wherein R^{r}, X, R¹, R², R^{t}, and stereochemical designations are as defined above for the compounds of Formulae (2), and R³ has the formula:

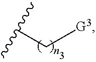

wherein n₁ is 0, 1, 2, 3, 4, 5, or 6, and G³ is H, C₁₋₆alkyl, C₃₋₈cycloalkyl, C₆₋₁₀aryl, C₁₋₁₀heteroaryl, OH, SH, SCH₃, COOH, CONH₂, NH₂, NHC(═NH)NH₂, COOH, CONH₂, or CH(OH)CH₃. In some embodiments, the flavor modifying compound is a compound of Formula (3a) or Formula (3b):

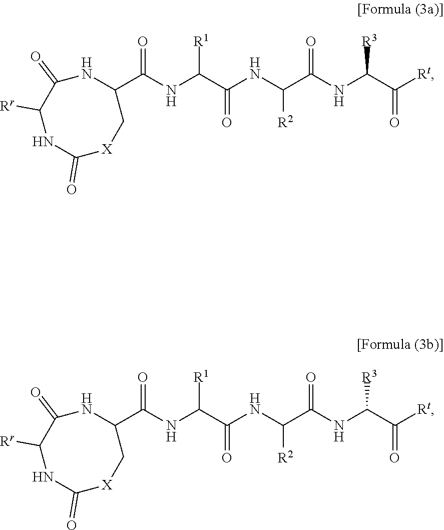

wherein the stereochemical configuration for the ring, R¹-bearing, and R²-bearing carbons are as defined for the compounds of Formulae (2a), (2b), (2c), (2d), (2e), (2f), (2g), (2h), (2i), (2j), (2k), (2l), (2m), (20), or (2p).

The peptidyl flavor modifying compounds disclosed herein may be combined with a variety of different consumables (e.g., foods and beverages). The compound(s) may be present in a concentration of no more than 100 mg/kg, no more than 50 mg/kg, no more than 25 mg/kg, no more than 10 mg/kg, no more than 5 mg/kg, no more than 2.5 mg/kg, no more than 1 mg/kg, no more than 0.5 mg/kg, no more than 0.1 mg/kg, no more than 0.05 mg/kg, no more than 0.01 mg/kg, no more than 0.005 mg/kg, or no more than 0.001 mg/kg. In the specific case of liquid consumables (e.g., beverages, soups, etc) the concentration of the compound(s) can be no more than 100 mg/L, no more than 50 mg/L, no more than 25 mg/L, no more than 10 mg/L, no more than 5 mg/L, no more than 2.5 mg/L, no more than 1 mg/L, no more than 0.5 mg/L, no more than 0.1 mg/L, no more than 0.05 mg/L, no more than 0.01 mg/L, no more than 0.005 mg/L, or no more than 0.001 mg/L. When a plurality of peptidyl compounds are present in the consumable, the disclosed concentrations refer to the sum total of all the peptidyl compounds.

In some embodiments, the compound(s) may be present in a concentration from 0.001-25 mg/kg, from 0.001-25 mg/kg, from 0.001-10 mg/kg, from 0.001-5 mg/kg, from 0.001-2.5 mg/kg, from 0.001-1 mg/kg, from 0.01-1 mg/kg, from 0.05-1 mg/kg, from 0.1-1 mg/kg, from 0.25-1 mg/kg, from 0.01-0.5 mg/kg, from 0.001-0.1 mg/kg, from 0.001-0.01 mg/kg, or from 0.01-0.1 mg/kg. In the specific case of liquid consumables, the compound(s) may be present in a concentration from 0.001-25 mg/L, from 0.001-25 mg/L, from 0.001-10 mg/L, from 0.001-5 mg/L, from 0.001-2.5 mg/L, from 0.001-1 mg/L, from 0.01-1 mg/L, from 0.05-1 mg/L, from 0.1-1 mg/L, from 0.25-1 mg/L, from 0.01-0.5 mg/L, from 0.001-0.1 mg/L, from 0.001-0.01 mg/L, or from 0.01-0.1 mg/L.

The flavor of a wide variety of consumables may be modified using the disclosed compounds. Exemplary foods and beverages include coffees, sodas, milk, tea, fruit juice, vegetable juice, vegetables, cheese, yogurts, grains, beers, wines, distilled spirits, cocoas, fruits, and vegetables, especially cruciferous vegetables.

In some embodiments, the consumable is a milk product, for example a cow milk, goat milk, sheep milk, plant milk. Exemplary plant milks include almond milk, coconut milk, soy milk, rice milk, hemp milk, oat milk, pea milk, and peanut milk.

In certain preferred embodiments, the flavor modifying compounds can be combined with a coffee product. In some embodiments, the consumable is a coffee product. As used herein, a coffee product can refer to both coffee beverages, e.g., brewed coffee, coffee concentrate, bottled coffee and the like. Coffee products also include coffee beans and processed coffee beans (raw, unroasted, roasted, fermented, ground, instant coffee products).

The perceived quality of a cup of coffee is impacted both by the specific flavors in the coffee, as well as the aroma of the coffee. Differences in aroma and flavor are perceived by the olfactory system. Differences in flavor are perceived in the gustation system, in which compounds interact with taste bud pores in the alimentary system. Differences in flavor are perceived by the somatosensory system, which can occur anywhere in the body, relating to or denoting sensations such as pressure, pain, or temperature. Chemesthesis is the direct activation of somatosensory nerves by chemical stimuli. The combination of these senses contributes to the overall perceived quality of the coffee. The quality of a particular cup of coffee can be assessed using the Cup Score system, established by the Specialty Coffee Association (“SCA”), and determined by one or more certified industrial Q graders. The maximum cup score is 100. Coffee can be broken in to two general categories based on cup score of ‘sub-specialty’ (less than 80) and ‘specialty coffee’ (equal to or greater than 80); within the specialty coffee, further categories are designated with increasing cup score, such as very good specialty (80-84.99), excellent specialty (85-89.99), and outstanding specialty (90-100) according to SCA cupping method; generally commercial samples in North America range in cup score between 75-90. Three quality groups based on cup score were assigned: high quality coffee has a cup score greater than 85, medium quality coffee has a cup score between 80-84.99, and low-quality coffee has a cup score less than 80.

In certain embodiments, the compounds disclosed herein may be added to an already brewed cup of coffee to enhance its quality. As used herein, a brewed coffee refers both to a beverage directly prepared from coffee grounds and consumed immediately thereafter, as well as coffee products which are packaged and sold as shelf-stable products. For instance, a low-quality coffee may be converted to a medium or high-quality coffee, a medium quality coffee may be converted to a high quality coffee, and a high quality coffee may be further enhanced to have an even high cup score. In certain embodiments, the compounds disclosed herein may be added in order to enhance the cup score by at least 5%, at least 10%, at least 15%, and least 20%, or at least 25%, relative to the cup score of the starting coffee. In certain embodiments, the compounds may be added in to enhance the cup score by at least 1 point, at least 2 points, at least 4 points, and least 5 points, at least 6 points, at least 7 points, at least 8 points, or at 9 points %, relative to the cup score of the starting coffee. In further embodiments, the compounds disclosed herein may be added to give a final coffee having a cup score from 70-100, 75-100, 80-100, from 85-100, from 87.5-100, from 90-100, from 92.5-100, from 95-100, or from 97.5-100.

In some embodiments, the compounds disclosed herein may be added to coffee beans, fermented coffee bean, roasted coffee beans, or ground coffee beans in order to enhance the quality of a coffee cup obtained from said beans. For instance, a low-quality coffee bean may be converted to a medium or high-quality coffee bean, a medium quality coffee bean may be converted to a high quality coffee bean, and a high quality coffee bean may be further enhanced to provide an even high cup score. In certain embodiments, the compounds disclosed herein may be added to the beans in order to enhance the cup score of a coffee cup obtained therefrom by at least 5%, at least 10%, at least 15%, and least 20%, or at least 25%, relative to the cup score of the coffee. In further embodiments, the compounds disclosed herein may be added to give a final coffee having a cup score from 80-100, from 85-100, from 87.5-100, from 90-100, from 92.5-100, from 95-100, or from 97.5-100.

The flavor modifying compounds may be added to coffee beans or grounds to increase the concentration of the compounds relative to unadulterated coffee beans or grounds. In some embodiments, the disclosed compounds can be added to soluble (i.e., instant) coffee compositions in similar amounts.

The compounds may be added to coffee beans, grinds and/or soluble (i.e., instant) coffee compositions in a variety of different manners. In some instances, the compounds may be directly admixed with dry beans, grinds and/or soluble coffee compositions in the concentrations described above. In other embodiments, the compounds may be dissolved or dispersed in a solvent, either water or organic solvent, and then combined with the beans or grinds for a time sufficient to impart the desired concentration of compounds in the beans or grinds.

The compounds may be combined with coffee during various stages of its processing. For instance, the compounds may be added prior to roasting, during roasting, after roasting, prior to fermentation, during fermentation, after fermentation, prior to grinding, during grinding, after grinding, prior to brewing, during brewing, after brewing, or after brewing and drying.

Coffee beans, e.g., green coffee beans, can be fermented with any of Gram-negative bacteria, bacilli, yeasts and filamentous fungi, acetic acid bacteria and lactic acid bacteria in the presence of the compounds disclosed herein. In some embodiments, the compounds can be directly added to the fermentation broth.

In some embodiments, the compounds can be used to modify or improve the body of a coffee product, for example as measured by cup score. Specific attributes which may be modified and/or improved include chalkiness, thickness, mouthcoating, and astringency.

### Examples

The following examples are for the purpose of illustration of the invention only and are not intended to limit the scope of the present invention in any manner whatsoever.

Coffee was brewed using an automatic drip coffee machine (Keurig K475, Keurig Green Mountain, Waterbury VT, USA). Portioned coffee brew pods were stored at −40° C. prior to analysis. Coffee was brewed using nanopure water and the 8 oz setting on the automatic drip machine. Four coffees were made from different beans and SCA Q-grade cuppers evaluated the ‘body’ score, which are shown in Table 1.

TABLE 1
Average Q-Grade cupper body scores in
four commercially available coffees

| Coffee # | Average Cupper body score |
|---|---|
| 1 | 7.5 |
| 2 | 7.0 |
| 3 | 5.7 |
| 4 | 4.8 |

While tasting the coffees which had received four different coffee “body” scores by trained coffee evaluators, attributes were generated by the descriptive analysis panel. One of the attributes was ‘chalkiness’ and a reference was chosen by the panel to represent the characteristic sensation (Table 2).

TABLE 2
List of definitions, references, and sample evaluation
procedures for each tactile attribute

| Attribute | Definition | Reference | Evaluation Procedure |
|---|---|---|---|
| Chalkiness | The sensation | 1% (w/v) Jif ™ | Move sample around |
| | of dustiness | Peanut Powder in | mouth taking note of |
| | or particulate | reverse osmosis | the feeling of |
| | in the sample | (RO) water | dustiness or particulate |
| | | | within the sample. |

In preparation for sensory validation, ((2R,5S)-2-isobutyl-3,8,dioxo-1,4-diazocane-5-carbonyl)-L-tryptophyl-L-histidine was quantified using standard addition in both the high body coffee (body score=7.5), as well as in the lowest body coffee from the original sample set (body score=4.8) (Table 3).

TABLE 3
concentration of chalkiness compound isomer 1 and
2 in high body (HB) and low body (LB) coffee

| | | Concentration | |
|---|---|---|---|
| ((2R,5S)-2-isobutyl-3,8,dioxo- | MRM transition | (mg/L) | |
| 1,4-diazocane-5-carbonyl)-L- | [collision | HB | LB |
| tryptophyl-L-histidine | energy, eV] | Coffee | Coffee |
| Isomer 1 | 564.3 → 154.0 [30] | 0.366 | 0.03 |
| Isomer 2 | 564.3 → 154.0 [30] | 0.477 | 0.03 |

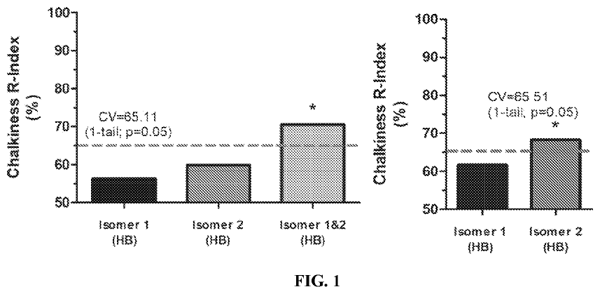

For validation in water, peptides were added to nanopure water at equivalent concentrations to that found in coffee sample 1. Similarly, for validation in coffee, peptides were added to coffee sample 4 (low body sample) to raise the concentration to be equivalent to coffee sample 1. Samples were then evaluated by 6 panelists in triplicate, with 3-4 samples presented per session. For evaluations in coffee, samples included isomer 1, isomer 2, combined isomer 1 & isomer 2, and a blind control (low body coffee). For evaluations in water, samples included isomer 1, isomer 2, and a blind control (water). Sample presentation order was randomized across panelists and replicates. For each evaluation, subjects were asked to indicate whether the sample was the same as the reference (water or low body coffee) and whether they were sure or unsure. Results were analyzed using an R-index methodology. R-indices are a measure of discrimination (O'Mahony, 1992). When implementing this methodology, blind controls are presented with the same frequency as stimulus samples, and this thereby enables comparison between the chance of a “correct hit” (i.e. calling the stimulus “different” from the reference) to the chance of a false alarm (i.e. calling the blind control “different”). Using this logic, the R-index can effectively be understood as a probability of a subject correctly discriminating between a stimulus and a control sample (O'Mahony 1992). Results from this analysis are displayed in FIG. 1.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1-93. (canceled)

94. A compound having the formula: 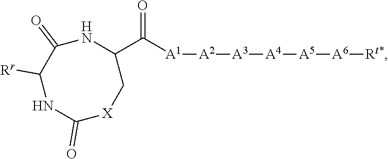
or a physiologically acceptable salt thereof, wherein:
R^{t*} is OH or NH₂;
X is null, CH₂, O, S, or Se;
R^{r} has the formula: 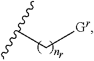
wherein nᵣ is 0, 1, 2, 3, 4, 5, or 6, and G^{r} is H, C₁₋₆alkyl, C₃₋₈ cycloalkyl, C₆₋₁₀aryl, C₁₋₁₀heteroaryl, OH, SH, SCH₃, COOH, CONH₂, NH₂, NHC(═NH)NH₂, COOH, CONH₂, or CH(OH)CH₃;
A¹ is an amino acid;
A² is an amino acid;
A³ is null or an amino acid;
A⁴ is null or an amino acid;
A⁵ is null or an amino acid; and
A⁶ is null or an amino acid.

95. The compound of claim 94, wherein the compound has the formula: 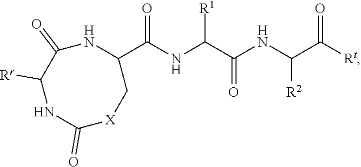
R^{t} is selected from-R^{t}*, -A³-R^{t}*, -A³-A⁴-R^{t}*, -A³-A⁴-R^{t}*, -A³-A⁴-A⁵-R^{t}*, and-A³-A⁴-A⁵-A⁶-R^{t}*,
R¹ has the formula: 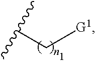
wherein n1 is 0, 1, 2, 3, 4, 5, or 6, and G¹ is H, C₁₋₆alkyl, C₃₋₈cycloalkyl, C₆₋₁₀aryl, C₁₋₁₀heteroaryl, OH, SH, SCH₃, COOH, CONH₂, NH₂, NHC(═NH)NH₂, COOH, CONH₂, or CH(OH)CH₃; and
R² has the formula: 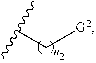
wherein n₂ is 0, 1, 2, 3, 4, 5, or 6, and G² is H, C₁₋₆alkyl, C₃₋₈cycloalkyl, C₆₋₁₀aryl, C₁₋₁₀heteroaryl, OH, SH, SCH₃, COOH, CONH₂, NH₂, NHC(═NH)NH₂, COOH, CONH₂, or CH(OH)CH₃.

96. The compound of claim 95, wherein the compound has the formula:

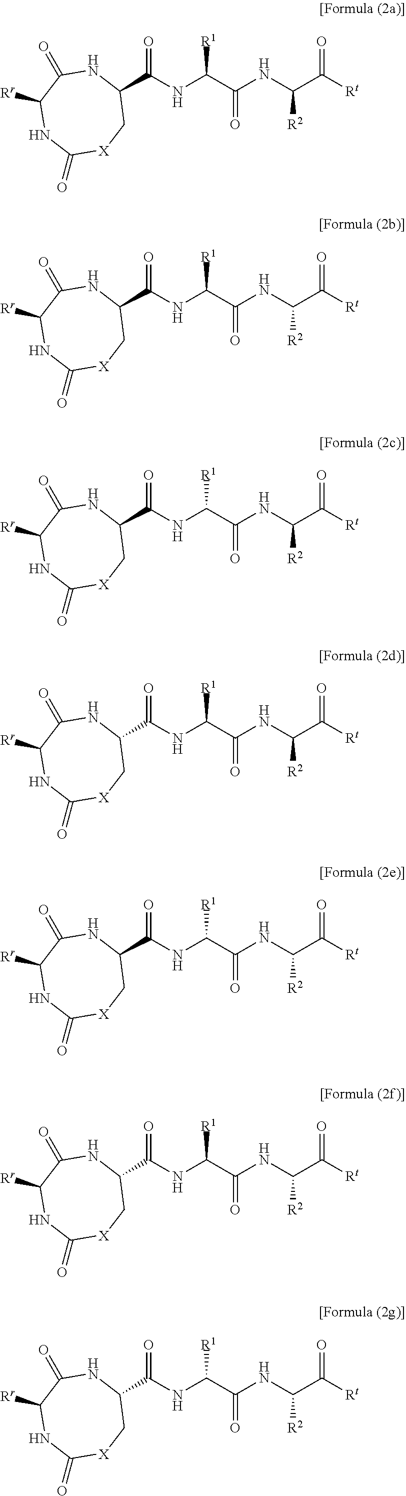

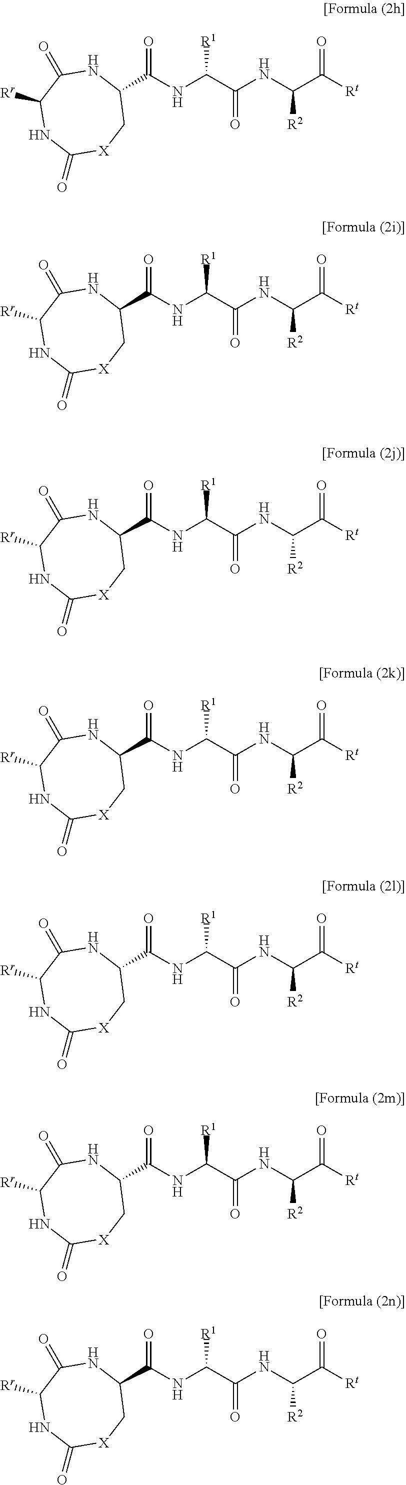

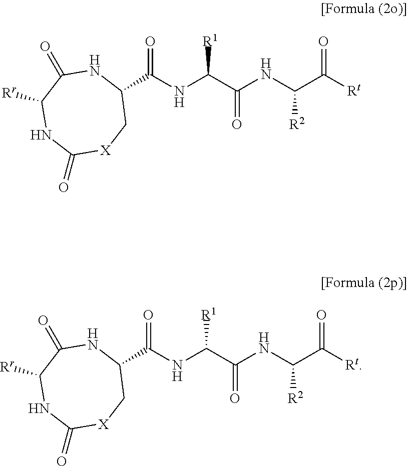

97. The compound of claim 95, wherein n is 0 or 1, and G^{r} is H, C₁₋₆alkyl, C₃₋₈cycloalkyl, C₆₋₁₀aryl, or C₁₋₁₀heteroaryl.

98. The compound of claim 95, wherein n is 0 and G^{r} is C₁₋₆alkyl.

99. The compound of claim 95, wherein R^{t} is R^{t*}.

100. The compound of claim 95, wherein n₁ is 1.

101. The compound of claim 95, wherein G¹ is H, C₁₋₆alkyl, C₃₋₈cycloalkyl, C₆₋₁₀aryl, or C₁₋₁₀heteroaryl.

102. The compound of claim 95, wherein n₂ is 1.

103. The compound of claim 95, wherein G² is H, C₁₋₆alkyl, C₃₋₈cycloalkyl, C₆₋₁₀aryl, or C₁₋₁₀heteroaryl.

104. The compound of claim 95, wherein R¹ has the formula:

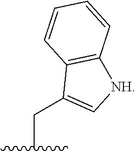 [SMILES: *Cc1c[nH]c2ccccc12]

105. The compound of claim 95, wherein R² has the formula:

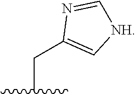 [SMILES: *Cc1c[nH]cn1]

106. The compound of claim 94, wherein X is null or CH₂.

107. The compound of claim 94, wherein R^{r} is H, methyl, or isopropyl.

108. A consumable product comprising one or more compounds of claim 94, wherein the one or more compounds are present in an amount of 0.001-25 mg/kg.

109. The consumable product of claim 108, wherein the consumable product is a coffee product or a milk product.

110. The consumable product of claim 109, wherein the consumable product is a milk product selected from cow milk, goat milk, sheep milk, almond milk, coconut milk, soy milk, rice milk, hemp milk, oat milk, pea milk, peanut milk, or a combination thereof.

111. A method for modulating the flavor of a consumable product, comprising adding one or more compounds of claim 94 to the consumable product.

112. The method of claim 111, wherein the one or more compounds are added in an amount of 0.001-25 mg/kg.

113. The method of claim 111, wherein the consumable product is a coffee product or a milk product.
